(19) United States
(12) Patent Application Publication
(10) Pub. No.: US 2025/0255925 A1
(43) Pub. Date: Aug. 14, 2025

(54) Topical Chemical Composition

(71) Applicant: Simply Roots, LLC, Indian Land, SC (US)

(72) Inventors: Mustafa F.KH. Khalaf, Amman (JO); Maurice G. Batshon, Waxhaw, NC (US)

(73) Assignee: Simply Roots, LLC, Indian Land, SC (US)

(21) Appl. No.: 19/047,687

(22) Filed: Feb. 7, 2025

Related U.S. Application Data
(60) Provisional application No. 63/551,583, filed on Feb. 9, 2024.

(51) Int. Cl. A61K 36/8962; A61K 9/00; A61K 31/01; A61K 31/19; A61K 33/00; A61K 36/752

(52) U.S. Cl. CPC A61K 36/8962; A61K 9/0014; A61K 31/01; A61K 31/19; A61K 33/00; A61K 36/752

(57) ABSTRACT

A topical cutaneous composition remarkably reduced eczema, psoriasis, and other skin problems in human patients. The topical cutaneous composition is formulated using natural and toxicologically harmless components as a skin cream with an acidic or a caustic property. When the topical cutaneous composition is applied to the afflisted area, the topical cutaneous composition attacks, sooths, and cures damaged skin.

### CROSS-REFERENCE TO RELATED APPLICATION

This patent application claims domestic benefit of U.S. Provisional Application No. 63/551,583 filed Feb. 9, 2024 and incorporated herein by reference in tits entirety.

### BACKGROUND

The subject matter described herein generally relates to medical preparations and, more particularly, the subject matter relates to medicinal chemical compounds and to skin care treatments.

Many people and pets suffer from skin conditions and disorders. Acne, eczema, psoriasis, dermatitis, rosacea, keratosis, cellulitis, warts, and blisters are just some of the common skin afflictions. Whatever the skin affliction, treatment is often slow, expensive, and time consuming.

### BRIEF DESCRIPTION OF THE SEVERAL VIEWS OF THE DRAWINGS

The features, aspects, and advantages of topical cutaneous composition are understood when the following Detailed Description is read with reference to the accompanying drawings, wherein:

FIG. 1 illustrates some examples of the topical cutaneous composition; and

FIG. 2 illustrates more examples of the topical cutaneous composition.

### DETAILED DESCRIPTION

The exemplary embodiments will now be described more fully hereinafter with reference to the accompanying drawings. The exemplary embodiments may, however, be embodied in many different forms and should not be construed as limited to the embodiments set forth herein. These embodiments are provided so that this disclosure will be thorough and complete and will fully convey the exemplary embodiments to those of ordinary skill in the art. Moreover, all statements herein reciting embodiments, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

As used herein, the singular forms “a,” “an,” and “the” are intended to include the plural forms as well, unless expressly stated otherwise. It will be further understood that the terms “includes,” “comprises,” “including,” and/or “comprising,” when used in this specification, specify the presence of stated features, integers, steps, operations, elements, ingredients, and/or components, but do not preclude the presence or addition of one or more other features, integers, steps, operations, elements, ingredients, and/or groups thereof. As used herein, the term “and/or” includes any and all combinations of one or more of the associated listed items.

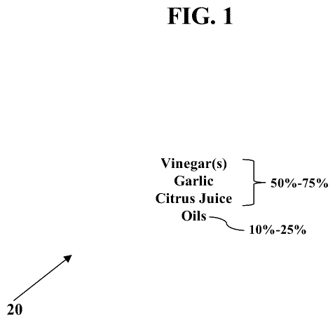

FIG. 1 illustrates examples of a topical cutaneous composition 20. While the topical cutaneous composition 20 may have other consistencies or thicknesses, the topical cutaneous composition 20 is a balm, cream, lotion, serum, or gel. The topical cutaneous composition 20 is applied/rubbed on and/or into the afflicted area of the skin. The topical cutaneous composition 20 is a mixture having anti-inflammatory, antibacterial, antiviral, and antioxidant activities. The topical cutaneous composition 20 is especially effective as a skin care product that attacks and promotes healing of wounds, burns, and dermatological conditions/diseases (such as eczema, psoriasis, and other skin problems).

Testing shows unexpected results. The topical cutaneous composition 20 has been extensively tested on about 250 humans. Of these patients, about sixty percent (60%) were women and about forty percent (40%) were men. The topical cutaneous composition 20 was applied to the females at their respective afflicted areas (such as faces, chests, breasts, and genital areas). The topical cutaneous composition 20 was applied to the males at their respective afflicted areas (such as legs, faces, ears, hands, and feet). Due to patient privacy concerns, this disclosure declines to provide photos and other treatment details. However, the results were exceptional, and all patients consistently reported extremely effective results, especially due to their sensitivity.

The topical cutaneous composition 20 may be applied to skin of humans and animals. The topical cutaneous composition 20, however, may be orally ingested. The topical cutaneous composition 20, for example, may be produced, containerized, and sold as a topical skin care cream for cutaneous ailments, such as eczema and psoriasis. The topical cutaneous composition 20, however, may also be formed as tablets or packaged as capsules for oral consumption. The topical cutaneous composition 20 may have an acidic mixture or blend of vinegar(s) (such as apple cider, red/white wine, balsamic, rice, malt, and/or champagne), garlic (perhaps crushed/powdered/paste/fresh), and citrus juice (e.g., lemon, lime, orange) having a concentration ranging from 50% to 75% percent of the total formulation value (such as w/w). The remainder of the ingredients (such as oily mixtures of extra virgin olive oil, peppermint oil and, if present, mineral/petroleum jelly) have 10% to 25% of the total formulation value (w/w). The peppermint oil mainly provides a pleasing fragrance, so other oils or fragrants may be substituted to suit personal needs and/or to provide product variety. Water, if needed, may be used to make up 100% of the total weight. An ointment mill, for example, may be used to forcibly spin, press, or otherwise mix the all-natural, vegan, editable, and/or herbal ingredients (whether liquid, powder, or paste) together to make them more homogenous. An undulator, as another example, when it mixes, it mixes, for example, at a speed range of 5,000-30,000 RPMs. The medicinal chemical compound may thus be smoothly rendered using machine-mixing/compounding, thus producing a professional and aesthetically beautiful product.

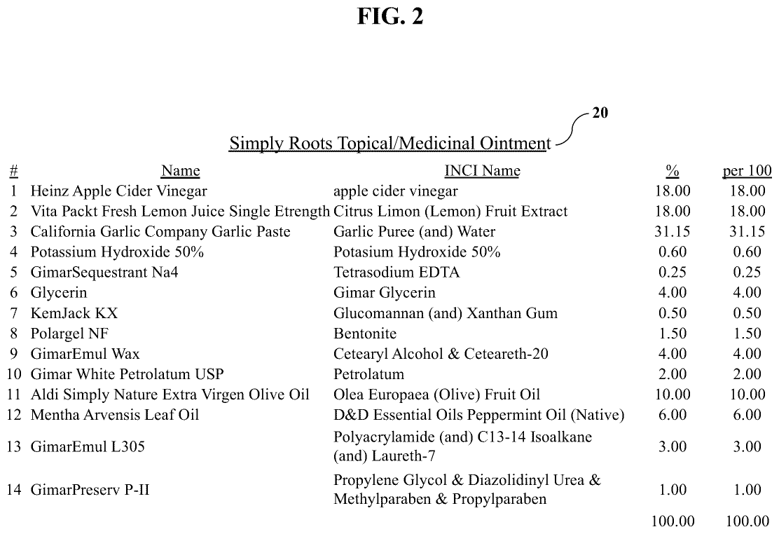

FIG. 2 illustrates more examples of the topical cutaneous composition 20. Here the topical cutaneous composition 20 was batch produced for production evaluation and additional patient testing. The topical cutaneous composition 20 is about 67% water, apple cider vinegar, garlic puree, and lemon fruit extract. The topical cutaneous composition 20 is neutralized using a potassium hydroxide (e.g., KOH or base/caustic potash). The potassium hydroxide may be added at a small amount (e.g., less than 1% w/w) to slightly increase pH and to reduce acidity. The potassium hydroxide, however, may be added at larger amounts (e.g., 5%-20% w/w or more) to increase alkalinity into slightly/mildly caustic pH. Cosmetic formulations with pH values lower than 3.5 may be at risk of safety issues. Such acid pH is due to the high levels of the apple cider vinegar and lemon juice, both acid materials, and the pH was adjusted with the addition of potassium hydroxide (KOH) as a pH modifier agent. The potassium hydroxide thus reacts with the acidic components to very slightly (perhaps acidly or caustically) burn, eat, or corrode the local skin area. The topical cutaneous composition 20 thus hastens decomposition of the surface of the afflicted/infected skin. The potassium hydroxide also reacts with the organic acidic components, thus converting skin oils (or fat) at the skin area to soap and glycerol. The topical cutaneous composition 20 may thus be a potassium soap that also cleanses, disinfects, and smooths the afflicted/infected skin.

The topical cutaneous composition 20 may have other components. A relatively small amount (e.g., less than 1% w/w) of tetrasodium EDTA, for example, may be added as a chelator that increases the usable shelf life of the topical cutaneous composition 20. Tetrasodium EDTA, as a salt of the acidic components, may also attack and decompose the surface of the afflicted/infected skin. Tetrasodium EDTA is a chelating/sequestering agent responsible for capturing free iron, calcium, and magnesium ions during production, and the unrestricted presence of these elements can compromise the stability of the cosmetic topical cutaneous composition 20. A small amount (e.g., about 2-6% w/w) of glycerin (glycerol), may be added as a surfactant and lubricant to ease rubbing application to the skin. The glycerin/glycerol also acts as a water-absorbing humectant to keep the topical cutaneous composition 20 moist. Then, when the topical cutaneous composition 20 is applied to the skin, the glycerin/glycerol also absorbs humidity in the air to help keep the skin moist. A relatively small amount (e.g., less than 1% w/w) of glucomannan and/or xanthan gum may be added as natural emulsifiers, thickeners, binders, and stabilizers, thus increasing the viscosity of the topical cutaneous composition 20.

The addition of water allows the introduction of a polymer as a rheology modifier, capable of suspending the garlic particles, taking into consideration that the raw material is “garlic puree,” meaning that the cosmetic topical cutaneous composition 20 needs to suspend many small particles of garlic homogenously. If the formulation omits a rheology modifier, such garlic particles will precipitate to the bottom of the jar/container. The ingredient acting as the rheology modifier is the blend of glucomannan and/or xanthan gum.

The topical cutaneous composition 20 may have other components. Bentonite clay may be added (such as 0%-3% w/w) as base fillers/binders to absorb liquids (such as the water) as thickening agents. Bentonite may also aid in decolorizing/clarifying the vinegar/acidic/oily components. Bentonite may also help increase the usable shelf life of the topical cutaneous composition 20. Bentonite thus helps deliver a cream/ointment form and works as a formula stabilizer. Cetearyl alcohol and/or ceteareth-20 may be added (such as 2%-6% w/w) as a surfactant and emulsifying agents to increase the viscosity of the topical cutaneous composition 20. Cetearyl alcohol and/or ceteareth-20 thus also protect, moisturize, and lubricate the afflicted/infected skin. Cetearyl alcohol and/or ceteareth-20 is a fatty alcohol with different functions, such as moisturizer and emollient, and it helps to stabilize the formulation. Cosmetic, hydrocarbonic petroleum jelly (or mineral oil) may be added (such as 0%-10%) as a lubricating, moisturizing, soothing skin protectant. Various oil blends (such as olive oil and/or peppermint oil) may be added (such as 10%-20%) as additional surfactants and emulsifying agents to increase the viscosity and to protect, moisturize, and lubricate the afflicted/infected skin.

The topical cutaneous composition 20 may have other components. Polyacrylamide and/or C13-14 isoalkane and/or laureth-7 is/are multifunctional polymer emulsifier mixtures that may be added (such as 0%-6% w/w) to increase viscosity. Polyacrylamide and/or C13-14 isoalkane and/or laureth-7 is an emulsifier blend that forms the emulsion with olive oil, petrolatum, and mint oil. Propylene glycol with diazolidinyl urea, methylparaben, and propylparaben may be added (such as 0%-4% w/w) as cosmetic preservatives with antimicrobial properties, thus also cleansing and disinfecting the afflicted/infected skin area.

The topical cutaneous composition 20 is thus a superior formulation. The topical cutaneous composition 20 may be a mixture of all natural components. The topical cutaneous composition 20 is formulated using toxicologically harmless components. The topical cutaneous composition 20 is a novel, acidic (e.g., pH≤5) and/or caustic (e.g., pH≥9) ointment or cream that attacks, sooths, and cures damaged skin. Initial, cosmetic testing shows the topical cutaneous composition 20 quickly and remarkably promotes reduced eczema, psoriasis, and other skin problems, thus allowing the patients to resume normal/public activities in shorter time.

1. A topical cutaneous composition, comprising:
a garlic and vinegar blend at 50-75% (w/w);
a natural oil at 10-25% (w/w);
a salt added at less than 1% (w/w); and
water added to the topical cutaneous composition to make 100% by weight.

2. The topical cutaneous composition of claim 1, further comprising a petrolatum.

3. The topical cutaneous composition of claim 1, further comprising a binder.

4. The topical cutaneous composition of claim 1, further comprising a sodium hydroxide.

5. The topical cutaneous composition of claim 1, further comprising a lemon juice.

6. The topical cutaneous composition of claim 1, further comprising a bentonite.

7. The topical cutaneous composition of claim 1, further comprising a wax.

8. The topical cutaneous composition of claim 1, further comprising an olive oil.

9. The topical cutaneous composition of claim 1, further comprising a peppermint oil.

10. The topical cutaneous composition of claim 1, further comprising a propylene glycol.

11. A topical cutaneous composition, comprising:
a garlic, vinegar, and potassium hydroxide blend at 50-75% (w/w);
a natural oil at 10-25% (w/w);
a salt added at less than 1% (w/w); and
water added to the topical cutaneous composition to make 100% by weight.

12. The topical cutaneous composition of claim 11, further comprising a petrolatum.

13. The topical cutaneous composition of claim 11, further comprising a binder agent.

14. The topical cutaneous composition of claim 11, further comprising an emulsifying agent.

15. The topical cutaneous composition of claim 11, further comprising a moisturizing agent.

16. A topical cutaneous composition, comprising:
a garlic, vinegar, lemon, and potassium hydroxide blend at 50-75% (w/w);
a tetrasodium EDTA at less than 1% (w/w);
a glycerin at 4% (w/w);
a glucomannan binder at at less than 1% (w/w);
a bentonite at less than 2% (w/w);
a cetearyl alcohol at 4% (w/w);
a petrolatum at 2% (w/w);
an olive oil at 10% (w/w);
a peppermint oil at 6% (w/w); and
water added to the topical cutaneous composition to make 100% by weight.

17. The topical cutaneous composition of claim 16, further comprising a caustic pH combination of the garlic, vinegar, lemon, and potassium hydroxide blend at 50-75% (w/w).
